(19) United States
(12) Patent Application Publication
(10) Pub. No.: US 2008/0039735 A1
(43) Pub. Date: Feb. 14, 2008

(54) Respiratory monitor display

(76) Inventor: Barry L. Hickerson, Modesto, CA (US)

Correspondence Address: LAW OFFICE OF GLENN R. SMITH, 28626 BROOKHILL ROAD, TRABUCO CANYON, CA, 92679-1163, US

(21) Appl. No.: 11/810,964

(22) Filed: Jun. 6, 2007

Related U.S. Application Data
(60) Provisional application No. 60/811,524, filed on Jun. 6, 2006.

(51) Int. Cl. A61B 5/08

(52) U.S. Cl. 600/532; 128/200.24

(57) ABSTRACT

A respiratory monitor measures expiratory carbon dioxide. A display has a measurement indicator combined with a color indicator. The measurement indicator corresponds to a measured value of carbon dioxide. The color indicator corresponds to a predetermined range of carbon dioxide values including the measured value so as to provide a readily recognized visual cue that carbon dioxide is in a normal range or an abnormal range.

### CROSS-REFERENCE TO RELATED APPLICATIONS

The present application claims priority benefit under 35 U.S.C. § 119(e) to U.S. Provisional Patent Application Ser. No. 60/811,524, filed Jun. 6, 2006, entitled “Respiratory Monitor Display,” incorporated herein by reference.

### BACKGROUND OF THE INVENTION

Respiratory carbon dioxide (CO₂) is an important parameter that provides valuable patient status information to a healthcare provider. A typical carbon dioxide monitor, or capnometer, analyzes carbon dioxide from respiratory airflow, either mainstream or sidestream, from an intubated or non-intubated patient. Mainstream monitors insert a sample chamber within the patient's gas flow, whereas sidestream monitors deliver a sample of gas flow to a remote sample chamber. Capnometers utilize, for example, optical means (infrared spectra analysis) or chemical means (pH analysis) for carbon dioxide measurements. Carbon dioxide is conventionally measured at “end-tidal” or the completion of the exhalation cycle. This end-tidal carbon dioxide measurement (ETCO₂) is a partial pressure expressed as millimeters of mercury (mmHg or Torr), a percentage of normal atmospheric pressure (760 mmHg at an ambient temperature of 25° C.), where 1%=0.01×760 mmHg=76 mmHg, or in kilo Pascals (kPa), where 1 kPa=7.5 mmHg. ETCO₂ is a non invasive measurement that approximates the partial pressure of carbon dioxide in the arterial blood (PaCO₂), where the normal gradient between the two measurements is about 5 to 6 mmHg. Normal ETCO₂ is in a range of about 35 to 45 mmHg or roughly 5% to 6% CO₂.

### SUMMARY OF THE INVENTION

Capnography has become a standard of care in surgical departments for airway and ventilation assessment. As examples, the absence of ETCO₂ may indicate esophageal intubation, accidental tracheal extubation, disconnection of the breathing circuit or complete obstruction of the endotracheal tube. In addition, capnography provides feedback for adjusting ventilator settings.

It is crucial for a monitor to quickly alert a practitioner of abnormal CO₂ measurements and allow ready determination of normal CO₂ measurements. A respiratory monitor display advantageously provides an ETCO₂ measurement in combination with a ETCO₂ range indicator. The range indicator allows quick visual recognition of a normal or abnormal ETCO₂ measurement. In an embodiment, the ETCO₂ range indicator is a color cue, such as green for normocarbia, and yellow or red for increasing degrees of hypocarbia or hypercarbia.

One aspect of a respiratory monitor display is a numerical indicator corresponding to a person's measured value of respiratory carbon dioxide and a color indicator also corresponding to the measured value of respiratory carbon dioxide. The color indicator is configured to display colors corresponding to each of a normal range of expiratory carbon dioxide and an abnormal range of respiratory carbon dioxide. In an embodiment, the numerical indicator is a number digit and the color indicator is a color applied to the digit itself or to the digit's background. In an embodiment, respiratory carbon dioxide is indicated with a bar graph. The numerical indicator is a printed number at the end of the bar. The color indicator is a color applied to the bar, where the bar may be continuous or a series of discrete LEDs. In an embodiment, the color indicator has a first color, such as a shade of green, for a normal range of ETCO₂, a second color, such as a shade of yellow, for a moderately abnormal range of ETCO₂, and a third color, such as a shade of red, for a significantly abnormal range of ETCO₂.

Another aspect of a respiratory monitor display comprises measuring respiratory carbon dioxide of a patient and displaying an end tidal carbon dioxide (ETCO₂) value from the measurement. A range of ETCO₂ is indicated in association with the displayed value. The range indication has at least a first color corresponding to a normal range of ETCO₂ and at least a second color corresponding to an abnormal range of ETCO₂. In an embodiment, a waveform of respiratory carbon dioxide versus time is displayed and coloring of at least a portion of the waveform indicates the ETCO₂ range. For example, the waveform plateau may be colored a shade of green when ETCO₂ is within a normal range of between about 35 mmHg and about 45 mmHg and colored a shade of from yellow to red when ETCO₂ is outside a normal range. In an embodiment, digits representing the ETCO₂ value are displayed and either the digits or the digit background are colored according a normal or abnormal ETCO₂ range. In another embodiment, a bar graph having a bar representing the ETCO₂ value is displayed and the bar is colored according to a normal or abnormal ETCO₂ range. The bar may be a continuous illumination or a series of discrete color indicators, such as LEDs, where at least one of the LEDs is illuminated in a color according to a normal or abnormal ETCO₂ range. In an embodiment, the bar is a shade of green when ETCO₂ is within a range of between about 35 mmHg and about 45 mmHg and a shade of from yellow to red when ETCO₂ is outside that normal range.

A further aspect of a respiratory monitor display comprises a measurement indicating means and a color indicating means. The measurement indicating means convey a measured value of a person's respiratory carbon dioxide. The color indicating means corresponds to the measurement indicating means and provides an immediate visualization or cue of a range of the measured value of respiratory carbon dioxide. A combination means combines the measurement indicating means and the color indicating means. In an embodiment, the combination means comprises a bar means for indicating the measured value and a bar color means for indicating the range. In an embodiment, the combination means comprises a digit means for indicating the measured value and a digit color means for indicating the range. In an embodiment, the color indicating means comprises a safe color means for indicating a normal range of measured ETCO₂ and a caution color means for indicating an abnormal range of measured ETCO₂.

### BRIEF DESCRIPTION OF THE DRAWINGS

FIG. 1 is an illustration of a respiratory monitor display combining a respiratory waveform and an ETCO₂ range indicator;

FIGS. 2A-C are illustrations of respiratory monitor displays combining a numerical ETCO₂ measurement and an ETCO₂ range indicator;

FIG. 3 is an illustration of a respiratory monitor display combining a continuous ETCO₂ bar graph and an ETCO₂ range indicator; and

FIG. 4 is an illustration of a respiratory monitor display combining a discrete ETCO₂ bar graph and an ETCO₂ range indicator.

### DETAILED DESCRIPTION OF THE PREFERRED EMBODIMENTS

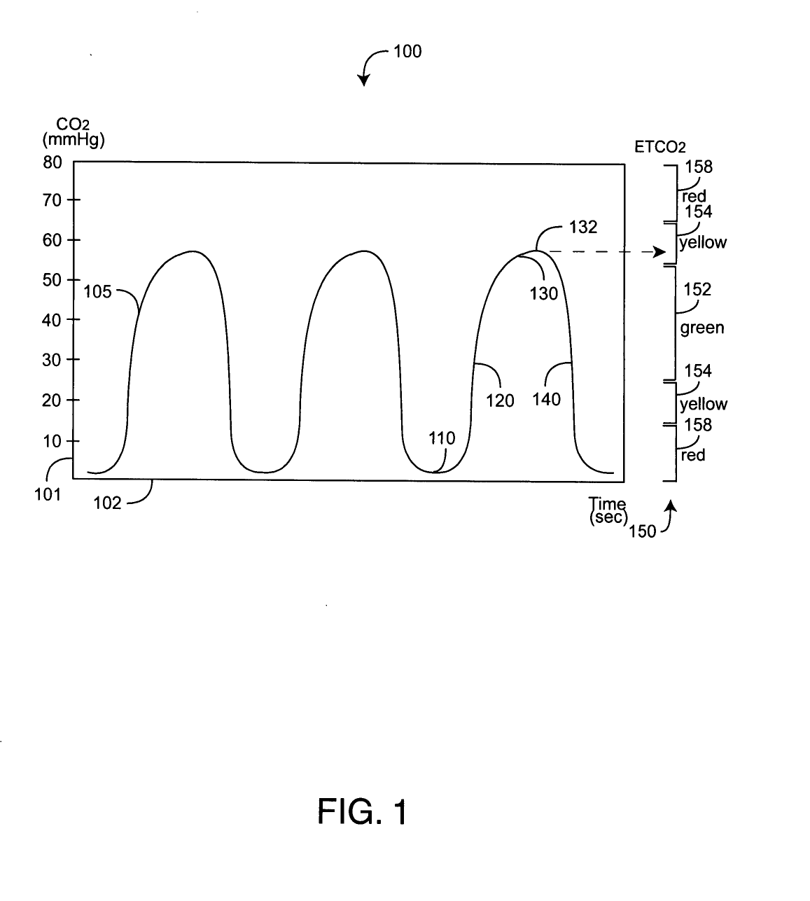

FIG. 1 illustrates a respiratory monitor display 100 having a CO₂ axis 101, a time axis 102 and a respiratory waveform 105. The waveform 105 has four phases corresponding to a respiratory cycle. The first phase occurs during inspiration. The second phase is the onset of expiration, which results in a rapid increase in CO₂. The third phase occurs as the CO₂ is exhaled from all the alveoli. The fourth phase is the onset of inspiration. These phases are delineated by an inspiratory baseline 110, an expiratory upstroke 120, an expiratory plateau 130 and an inspiratory downstroke 140. The highest point 132 of the plateau 130 is the ETCO₂ measurement at the end of expiration. In an embodiment, the waveform 105 is displayed in a color associated with one of a predetermined set of ETCO₂ ranges 150. In an embodiment, the respiratory waveform 105 is a green shade when ETCO₂ is in a normal range 152, a yellow shade when ETCO₂ is moderately outside of a normal range 154 and a red shade when ETCO₂ is significantly outside a normal range 158. In an embodiment, only portions of the waveform trace are colored according to a measured ETCO₂ range, such as the plateau 130.

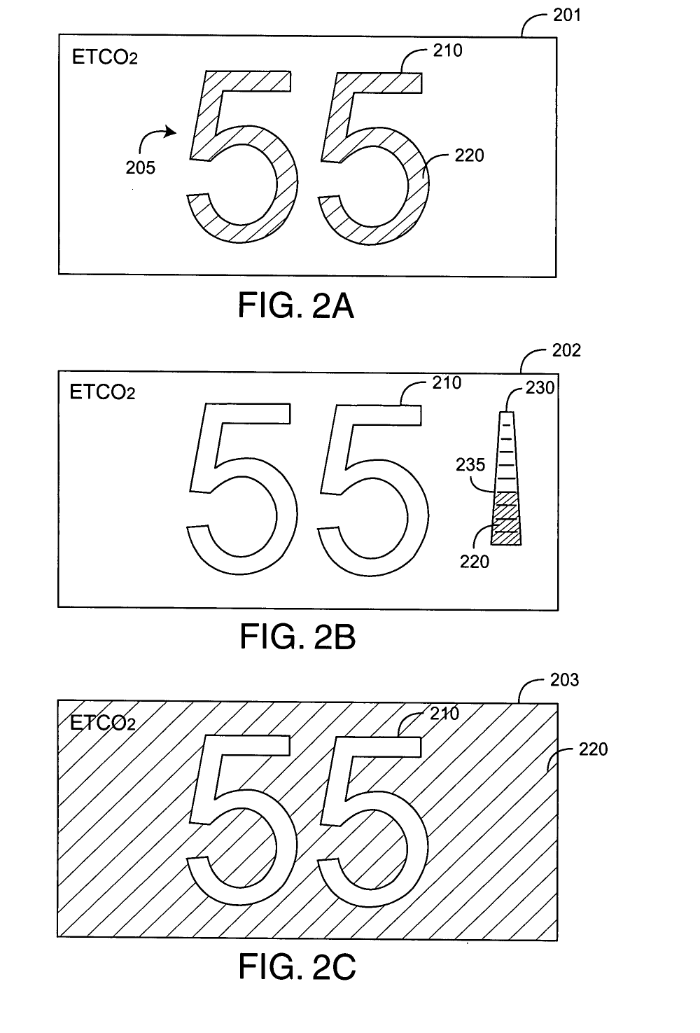

FIGS. 2A-C illustrate respiratory monitor displays 201-203 having numbers 210 representing an ETCO₂ measurement and associated color indicators 220 representing an ETCO₂ range. FIG. 2A shows a respiratory monitor display 201 embodiment having colored digits 205, where the digits 210 represent the numerical value of an ETCO₂ measurement and the color of the digits 220 indicates an ETCO₂ range. In an embodiment, the colored digits 205 are a green shade when indicating a normal ETCO₂ range, a yellow shade when indicating a moderately abnormal ETCO₂ range and a red shade when indicating a significantly abnormal ETCO₂ range.

As shown in FIG. 2B, a respiratory monitor display 202 has digits 210 and a vertical bar graph 230. As above, the digits 210 represent the numerical value of an ETCO₂ measurement. The height 235 of the bar graph 230 also corresponds to the numerical value of the ETCO₂ measurement. Further, the color 220 of the bar graph 230 to its measured height 235 indicates an ETCO₂ range. In an embodiment, the bar graph color 220 is a green shade when indicating a normal ETCO₂ range, a yellow shade when indicating a moderately abnormal ETCO₂ range and a red shade when indicating a significantly abnormal ETCO₂ range.

As shown in FIG. 2C, a respiratory monitor display 203 has digits 210 displayed over a colored background 220. As above, the digits 210 represent the numerical value of an ETCO₂ measurement. The background color 220 indicates an ETCO₂ range. In an embodiment, the background is a green shade when ETCO₂ is in a normal range, a yellow shade when ETCO₂ is in a moderately abnormal ETCO₂ range and a red shade when ETCO₂ is in a significantly abnormal range.

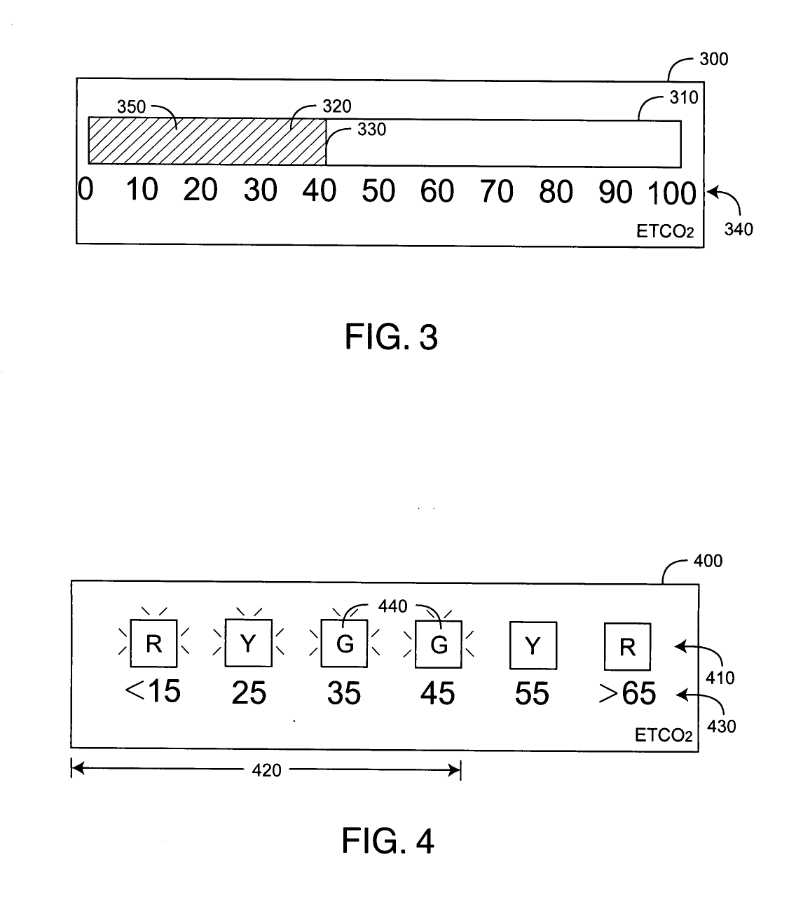

FIG. 3 illustrates a respiratory monitor display 300 having a continuous colored bar graph 310. In an embodiment, the length of the colored bar 320 indicates an ETCO₂ measurement. In an embodiment, printed numbers 340 aligned with the bar end 330 designate an ETCO₂ measurement value. A color indicator 350 along the length of the bar 320 corresponds to one or more ETCO₂ ranges. In an embodiment, at least a portion of the bar is a green shade corresponding to a normal ETCO₂ range, a yellow shade corresponding to a moderately abnormal ETCO₂ range and a red shade corresponding to a significantly abnormal ETCO₂ range.

FIG. 4 illustrates a respiratory monitor display 400 having a discrete colored bar graph 410 having an illuminated bar 420, printed numbers 430 and color range indicators 440. In an embodiment, the bar is composed of multiple LEDs (light emitting diodes), where a single LED is illuminated corresponding to a particular ETCO₂ measurement and corresponding printed number. In an embodiment, multiple LEDs are illuminated along the bar length 420, the last of which corresponds to a particular ETCO₂ measurement. In an embodiment, the LEDs emit light having a color corresponding to an ETCO₂ range. For example, the bar is composed of green (G) LEDs corresponding to a normal ETCO₂ range, yellow (Y) LEDs corresponding to a moderately abnormal ETCO₂ range and red (R) LEDs corresponding to a significantly abnormal ETCO₂ range, as shown.

In various embodiments described above, the colors green, yellow and red, and shades thereof, are used to represent normal, moderately abnormal and significantly abnormal ETCO₂ range intervals, respectively. For example, green is a color cue that something is “safe,” and yellow or red is a color cue for “caution.” In each of those embodiments, colors other than green, yellow and red may be used to represent those range intervals; different range intervals may be designated by these colors; more range intervals may be represented by additional colors; colors representing hypocarbia range intervals may be different than colors representing hypercarbia range intervals; color combinations may be used to represent each interval; and multiple colors displayed simultaneously may represent particular range intervals, such as a bar graph that has progressively red, yellow, green, yellow and red sections displayed as ETCO₂ increases.

In various embodiments described above, a normal ETCO₂ range is considered to be between about 35 to 45 mmHg, a moderately abnormal ETCO₂ range is considered to be between about 25 to 35 mmHg or between about 45 to 55 mmHg, and a significantly abnormal ETCO₂ range is considered to be below about 25 mmHg or above about 55 mmHg. In various embodiments described above, a significantly abnormal ETCO₂ range is considered to be below about 15 mmHg and above about 65 mmHg, an abnormal ETCO₂ range is considered to be between about 15 mmHg to 25 mmHg and between about 55 mmHg to 65 mmHg, and a normal ETCO₂ range is considered to be between about 35 to 45 mmHg. Other embodiments may include different range intervals and more range intervals or both. In an embodiment, instead of LEDs, the bar of a bar graph is composed of chemical indicators that display a color in response to the pH of the respiratory gas flow.

In various embodiments described above, ETCO₂ range intervals are indicated by an associated color indicator. Other embodiments may include an audible range indicator in lieu of or in addition to a color range indicator. An audible range indicator may be one or more tones, sound volumes or sound patterns, or absence of one or more tones, volumes or patterns, which are triggered within various ETCO₂ range intervals so as to promptly alert a practitioner of an ETCO₂ measurement.

A respiratory monitor display has been disclosed in detail in connection with various embodiments. These embodiments are disclosed by way of examples only and are not to limit the scope of the claims that follow. One of ordinary skill in art will appreciate many variations and modifications.

What is claimed is:

1. A respiratory monitor display comprising:
a numerical indicator corresponding to a measured value of a person's respiratory carbon dioxide; and
a color indicator also corresponding to the measured value of respiratory carbon dioxide,
wherein the color indicator is configured to display at least one of a plurality of colors corresponding to each of at least a normal range of the measured value of respiratory carbon dioxide and an abnormal range of the measured value of respiratory carbon dioxide.

2. The respiratory monitor display according to claim 1 wherein the numerical indicator is at least one number digit and the color indicator is applied to the number digit.

3. The respiratory monitor display according to claim 1 wherein the numerical indicator is at least one number digit and the color indicator is applied to a background for the number digit.

4. The respiratory monitor display according to claim 1 further comprising:
a bar graph having a bar; and
a plurality of numbers printed along the bar graph,
wherein the numerical indicator is a particular one of the numbers aligned with the end of the bar, and
wherein the color indicator is applied to the bar.

5. The respiratory monitor display according to claim 4 wherein the bar is continuous.

6. The respiratory monitor according to claim 4 wherein the bar is a series of discrete LEDs.

7. The respiratory monitor display according to claim 1 wherein the color indicator comprises:
a first color for a normal range of ETCO₂;
a second color for a moderately abnormal range of ETCO₂; and
a third color for a significantly abnormal range of ETCO₂.

8. The respiratory monitor display according to claim 7 wherein the first color is green, the second color is yellow and the third color is red.

9. A respiratory monitor display method comprising:
measuring respiratory carbon dioxide of a person;
displaying an end tidal carbon dioxide (ETCO₂) value from the measurement; and
indicating a range of ETCO₂ in association with the displayed value,
wherein the range indication has at least a first color corresponding to a normal range of ETCO₂ and at least a second color corresponding to an abnormal range of ETCO₂.

10. The respiratory monitor display according to claim 9 wherein:
displaying comprises plotting a waveform of respiratory carbon dioxide versus time, and
indicating comprises coloring at least a portion of the waveform according to the ETCO₂ range.

11. The respiratory monitor display according to claim 10 wherein the indicating comprises:
coloring at least a portion of the waveform plateau a shade of green when ETCO₂ is within a range of between about 35 mmHg and about 45 mmHg; and
coloring at least a portion of the waveform plateau a shade of from yellow to red when ETCO₂ is outside the range of between about 35 mmHg and about 45 mmHg.

12. The respiratory monitor display according to claim 9 wherein:
displaying comprises presenting a plurality of digits representing the ETCO₂ value; and
indicating comprises coloring at least one of the digits and a background of the digits according to the ETCO₂ range.

13. The respiratory monitor display according to claim 9 wherein:
displaying comprises presenting a bar-graph having a bar representing the ETCO₂ value; and
indicating comprises coloring the bar according to the ETCO₂ range.

14. The respiratory monitor display according to claim 13 wherein coloring comprises:
providing a series of colored LEDs along the bar;
illuminating at least one of the LEDs according to the ETCO₂ range.

15. The respiratory monitor display according to claim 13 wherein the indicating comprises:
coloring at least a portion of the bar a shade of green when ETCO₂ is within a range of between about 35 mmHg and about 45 mmHg; and
coloring at least a portion of the bar a shade of from yellow to red when ETCO₂ is outside the range of between about 35 mmHg and about 45 mmHg.

16. A respiratory monitor display comprising:
a measurement indicating means for conveying a measured value of a person's respiratory carbon dioxide; and
a color indicating means corresponding to the measurement indicating means for providing immediate visualization of a range of the measured value of respiratory carbon dioxide.

17. The respiratory monitor display according to claim 16 further comprising a combination means for combining the measurement indicating means and the color indicating means.

18. The respirator monitor display according to claim 17 wherein the combination means comprises:
a bar means for indicating the measured value; and
a bar color means for indicating the range.

19. The respirator monitor display according to claim 17 wherein the combination means comprises:
a digit means for indicating the measured value; and
a digit color means for indicating the range.

20. The respiratory monitor display according to claim 16 wherein the color indicating means comprises:
a safe color means for indicating a normal range of measured ETCO₂; and
a caution color means for indicating an abnormal range of measured ETCO₂.
